(19) United States
(12) Patent Application Publication
(10) Pub. No.: US 2006/0286058 A1
(43) Pub. Date: Dec. 21, 2006

(54) System for cleaning and conditioning hair

(75) Inventors: Mrunalini Shireesh Dhamdhere, Des Plaines, IL (US); Keith Leslie Rutherford, Port Sunlight Village (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/154,407

(22) Filed: Jun. 16, 2005

(51) Int. Cl. A61K 8/73; A61K 8/89

(52) U.S. Cl. 424/70.12; 424/70.13

(57) ABSTRACT

A system for cleaning and conditioning hair, in particular, fine, thin hair is provided. The system comprises, as individually packaged compositions: (A) an aqueous pre-shampoo conditioner comprising: i) a cationic conditioning polymer having a charge density of at least 1.8 meq/g, ii) cationic conditioning surfactant, iii) a fatty alcohol material; and (B) an aqueous shampoo comprising: i) an anionic surfactant, and ii) a cationic conditioning agent selected from the group consisting of quaternary silicone polymers, quaternary polysaccharide polymers, quaternary cellulose derivatives, quaternary starch derivatives, quaternary guar gum derivatives and mixtures thereof. Methods for cleaning and conditioning hair using such aqueous pre-shampoo conditioners and shampoos are also disclosed.

### FIELD OF THE INVENTION

This invention relates to a system for cleaning and conditioning hair comprising, as individually packaged components thereof, a pre-shampoo conditioner and a shampoo.

### BACKGROUND OF THE INVENTION

Hair shampoos are generally formulated with highly effective anionic surfactants that primarily clean, as opposed to condition, hair. Anionic surfactants not only remove dirt and soil from the hair, but also remove lipids naturally present on the surface of the hair fibers. Absent the use of conditioning agents, thoroughly cleansed hair can be difficult to comb, in both the wet and the dry state, because the individual hair fibers tend to snarl, kink and tangle with each other. To compensate for the removal of these lipids, one or more conditioners are commonly applied to the hair through the use of conditioning shampoos and/or after-shampoo conditioners.

Conditioning shampoos are single formulations that provide the dual purpose of cleaning and conditioning. The level of conditioning provided by single formulation shampoo/conditioners may, however, be excessive and inappropriate for fine, thin hair. Additionally, the use of single formulation compositions may make it difficult to selectively condition limited areas of the hair, for example, ends.

Shampooing followed by the application of an after-shampoo conditioner may provide good conditioning performance in terms of providing good combability, both wet and dry, and a smooth, soft “feel”. With repeated use, after-shampoo conditioners may leave fine, thin diameter hair “limp” and unmanageable. After-shampoo rinses may also weigh down fine, thin hair giving it a matted, limp appearance, which can exacerbate the “volume” issues faced by these users.

It is also known to treat the hair with a conditioner prior to shampooing. Among the pre-shampoo conditioners that have been disclosed are compositions that contain quaternary ammonium conditioners.

U.S. Pat. No. 3,980,091, for example, discloses compositions which are applied to the hair prior to shampooing, which compositions comprise a readily water soluble quaternary ammonium compound such as, for example, octyltrimethyl ammonium chloride, decyltrimethyl ammonium chloride; lauryldimethylethyl ammonium chloride; dodecyltrimethyl ammonium chloride; tetradecyltrimethyl ammonium chloride; hexadecyltrimethyl ammonium chloride; octadecyltrimethyl ammonium chloride; octadecyltrimethyl ammonium chloride, and mixed higher alkyl trimethyl ammonium chlorides containing mixtures of predominately C₈-C₁₈ alkyl radicals.

U.S. Pat. No. 4,061,150 discloses compositions for the treatment of hair prior to shampooing with an anionic shampoo, which compositions comprise a major portion of water, a quaternary ammonium that is substantive to the hair and comprises a long chain aliphatic hydrocarbon radical directly or indirectly linked to a quaternary nitrogen atom. Illustrative examples of the quaternary ammonium compounds include octyltrimethyl ammonium chloride; decyltrimethyl ammonium chloride; dodecyldimethylethyl ammonium chloride; dodecyltrimethyl ammonium chloride; tetradecyltrimethyl ammonium chloride; hexadecyltrimethyl ammonium chloride; octadecyltrimethyl ammonium chloride; dodecyldimethylbenzyl ammonium chloride; tetradecyldimethylbenzyl ammonium chloride; octadecyldimethylbenzyl ammonium chloride; octylmethyldibenzyl ammonium chloride; mixed higher alkyl trimethyl ammonium chlorides containing mixtures of predominately C₈-C₁₈ alkyl radicals.

U.S. Pat. No. 4,597,962 discloses a cosmetic hair-care composition containing (a) at least one cationic surface active agent which can be dispersed in water, (b) at least one water-soluble quaternised polymer of the ionene type and (c) at least one cationic silicone polymer. This patent discloses that its cosmetic hair-care compositions may be applied in the form of shampoos, rinsing products to be applied before or after shampooing, before or after tinting or bleaching, or before or after permanent waving or straightening, products for setting or brushing, conditioning compositions, restoring compositions and compositions for permanent-waved hair.

U.S. Pat. No. 4,180,089 discloses the use of various quaternary ammonium salts in hair care preparations, such as shampoos, hair rinses, hair-restoratives, hair tints, hair lacquers, pre-shampoo hair conditioners, hair tonic, setting lotions, hairsprays, hair creams, hair gels, hair oils, hair pomades or brilliantines.

U.S. Pat. No. 4,402,936 discloses a preshampoo-type hair treatment composition which comprises a cationic polymer that is a polymeric derivative of a monomer containing a cyclic cationic group. Hompolymers or copolymers of a diallyl quaternary ammonium compound or a vinylpyridine cationic polymer are described as examples of the preferred cationic polymers.

U.S. Pat. No. 4,495,173 discloses a pre-shampoo type hair treatment composition which comprises a suspension or solution, in a polar solvent, of at least one decomposition derivative of keratin material as therein more particularly described and at least one cationic polymer. The cationic polymers include polymeric materials of diallyl quaternary ammonium salts, cationic celluloses, cationic starches, and cationic vinyl polymers.

U.S. Pat. No. 5,288,484 discloses a pre-shampoo conditioning composition comprising an aqueous system of from about 0.1 to 20 weight percent of a cationic cellulose derivative quaternized with fatty C₁₀₋₁₈ alkyl groups; from about 0.05 to about 20 weight percent of a quaternary polymer; and from about 0.02 to about 10 weight percent of a quaternary ammonium salt of the formula RN⁺(CH₃)₂CH₂CHOHCH₂OH X⁻ wherein R is an alkyl group of about 7 to about 24 carbon atoms or an alkylamido group as therein more particularly described. Polydiallyidimethylammonium chloride is disclosed as a preferred quaternary polymer.

Pre-shampoo conditioners vary in their ability to impart desired conditioning benefits to hair. Moreover, the performance of a pre-shampoo conditioner can be significantly affected by the shampoo with which it is used. An improved means for cleaning and conditioning hair, particularly fine, thin hair, is desired.

Accordingly, it is an object of the present invention to provide a hair cleaning and conditioning product that imparts a combination of good conditioning, volume and body properties to hair.

It is a further object of this invention to provide a hair cleaning and conditioning product that imparts body and fullness to the hair, particularly fine, thin hair, while leaving the hair feeling clean and non-greasy.

Another object of the present invention to provide a product that ensures that complimentary pre-shampoo conditioner and shampoo compositions are provided to the consumer as a single unit.

Yet another object of the present invention is to provide a product that assists the consumer in following a hair treatment regime that involves the use of separately applied pre-shampoo conditioner and shampoo compositions.

### SUMMARY OF THE INVENTION

The present invention relates to a system for treating and conditioning hair which system comprises, as individually packaged compositions:

(A) an aqueous pre-shampoo conditioner comprising:
i) cationic conditioning polymer having a charge density of at least 1.8 meq/g,
ii) cationic conditioning surfactant, and
iii) fatty alcohol material; and

(B) an aqueous shampoo comprising:
i) anionic surfactant, and
ii) cationic conditioning agent selected from the group consisting of quaternary silicone polymers, quaternary polysaccharide polymers, quaternary cellulose derivatives, quaternary starch derivatives, quaternary guar gum derivatives, and mixtures thereof.

The system of the subject invention has particular application to the treatment and conditioning of fine, thin hair, but may also be used to add volume and body to all types of hair. In a further embodiment, the present invention relates to a method for cleaning and conditioning hair that comprises the sequential steps of:

(A) treating the hair with an aqueous pre-shampoo conditioner comprising:
i) cationic conditioning polymer having a charge density of at least 1.8 meq/g,
ii) cationic conditioning surfactant,
iii) fatty alcohol material; and

(B) treating the hair with an aqueous shampoo comprising:
i) anionic surfactant and
ii) cationic conditioning agent selected from the group consisting of quaternary silicone polymers, quaternary polysaccharide polymers, quaternary cellulose derivatives, quaternary starch derivatives, quaternary guar gum derivatives, and mixtures thereof.

### DETAILED DESCRIPTION OF THE INVENTION

As used herein % or weight % refers to the percent by weight of an ingredient as compared to the total weight of the composition or component that is being discussed. Except in the operating and comparative examples, or where otherwise explicitly indicated, all numbers in this description indicating amounts of materials or conditions of reaction, physical properties of materials and/or use are to be understood as modified by the word “about”. All amounts are by weight of the final composition, unless otherwise specified.

It should be noted that in specifying any range of concentration, any particular upper concentration can be associated with any particular lower concentration.

For the avoidance of doubt, the word “comprising” is intended to mean “including” but not necessarily “consisting of” or “composed of”. In other words, the listed steps or options need not be exhaustive.

The cationic conditioning polymer used in the preshampoo conditioner of this invention is characterized by the presence of cationic nitrogen groups such as quaternary ammonium groups, protonated amino groups or mixtures thereof. The cationic nitrogen-containing group will generally be present as a substituent on a fraction of the total monomer units of the cationic polymer. Such polymers are described in the CTFA Cosmetic Ingredient Directory, 3^{rd} edition. The weight average molecular weight of the polymer will preferably range from about 10,000 to about 2,000,000, preferably from about 50,000 to about 1,000,000.

The charge density of the polymer results, in part, from the ratio of cationic to non-cationic monomer units. Cationic conditioning polymers having charge densities of at least 1.8 meq/g are found to impart desirable conditioning benefits to the pre-shampoo conditioners of this invention, with cationic conditioning polymers having charge densities of at least 2 meq/g, more particularly from 2 meq/g to 4 meq/g, being preferred. In at least one embodiment of this invention, the use of cationic conditioning polymers having a charge density of from 2 meq/g to 3 meq/g is of particular interest. In another embodiment, cationic polymers having a charge density of from 2 meq/g to 2.5 meq/g have been found to be especially useful. Charge densities may be calculated by anionic potentiometric titration testing, a description of which is provided in the Examples that follow.

Among the cationic conditioning polymers suitable for use in the practice of this invention are diallyl ammonium polymers having units of the formula: 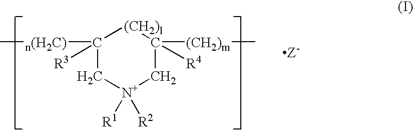
wherein R¹ and R², which may be the same or different, represent hydrogen or an alkyl group having 1-18, preferably 1-4 carbon atoms, R³ and R⁴, which may be the same or different, represent hydrogen, an alkyl group having 1-3 carbon atoms or a phenyl group, l and m and n are integers having a value of 0 or 1, with the proviso that at least one of m or n is 1; and Z is a salt-forming anion, preferably chloride or bromide. In one embodiment of the invention, when l is 0, both m and n are 1.

The polydiallylammonium polymer may be prepared, for example, by polymerizing diallyidimethylammonium chloride or bromide, or other suitable diallyldimethylammonium salts, using a free radical generating polymerization catalyst, such as a peroxide or hydroperoxide, then employing a suitable ion exchange resin, if desired, according to the methods described in U.S. Pat. Nos. 3,288,770 and 3,412,091. Exemplary of the polydiallylammonium salts used herein are polydiallyidimethylammonium chloride and polydiallyidimethylammonium bromide. A preferred polydiallylammonium salt is polydiallyidimethylammonium chloride, as recognized by the CTFA name of Polyquaternium-6.

Other suitable cationic conditioning polymers are polymers having units of the formula 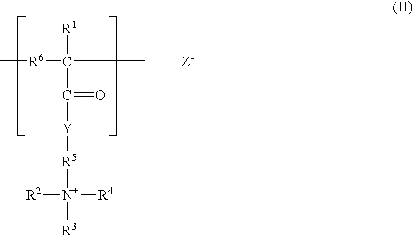
wherein R¹, stands for a hydrogen atom or an alkyl group having 1 to 3 carbon atoms, R², R³ and R⁴, which may be the same or different, stand for hydrogen, an alkyl group having 1 to 3 carbon atoms or a phenyl group, R⁵ is a divalent aliphatic hydrocarbyl group having from 2 to 6 carbon atoms, and R⁶ is a divalent aliphatic hydrocarbyl group having from 1 to 6 carbon atoms which is optionally substituted with ester and/or ester amide linkages, Y is O or N(R⁷) where R⁷ is a hydrogen atom or an alkyl group having 1 to 3 carbon atoms, and Z is a salt forming anion, preferably chloride or bromide. Exemplary of such polymers are, for example, ethanaminium, N,N,N-trimethyl-2-[(2-methyl-1-oxo-2-propenyl)oxy]-, chloride, homopolymer, as recognized by the CTFA name of Polyquaternium-32; and N,N,N-trimethyl-2-[(methyl-1-oxo-2-propenyl)oxy]-, chloride, homopolymer, as recognized by the CTFA name of Polyquaternium-37.

The cationic conditioning polymers may be formulated as homopolymers or copolymers. It should be appreciated that the term “homopolymers” includes polymers that contain both cationic and non-cationic forms of the above-described repeating units, i.e., not all of the repeating units are cationic. Non-limiting examples of comonomers from which the cationic conditioning copolymers may be formed are: (meth)acrylamide, alkyl and dialkyl(meth)acrylamide, alkyl(meth)acrylate, vinyl caprolactone and vinyl pyrrolidine. The alkyl and dialkyl substituted monomers typically have C1-C7 alkyl groups, with C1-C3 alkyl groups being of particular interest in many instances. Other suitable comonomers include, for example, vinyl esters, vinyl alcohols, maleic anhydride, propylene glycol and ethylene glycol. Cationic polymers suitable for use herein are commercially available from numerous suppliers under a variety of trade names.

The foregoing are intended as non-limiting examples of the cationic conditioning polymers suitable for use in the pre-shampoo conditioner of the subject invention. Desirably, the cationic conditioning polymers are present in the pre-shampoo conditioner in amounts of from 0.5 to 3 weight percent, preferably from 0.7 to 2.5 weight percent, and in an embodiment of particular interest, from 0.8 to 2.0 to weight percent, based on the total weight of the pre-shampoo conditioner. The amount of cationic conditioning polymer is exclusive of any suspending agents, solvents, or carriers present in such component, i.e., the amount of the cationic conditioning polymer is reported on an active basis.

The pre-shampoo conditioner of this invention further comprises at least one cationic conditioning surfactant. Cationic conditioning surfactants useful in the pre-shampoo conditioners of this invention contain amino or quaternary ammonium hydrophilic moieties which are positively charged when dissolved therein.

Examples of suitable cationic conditioning surfactants are those corresponding to the general formula:
[N(R¹)(R²)(R³)(R⁴)]⁺[X]⁻ (III)
wherein R¹, R², R³, and R⁴ are independently selected from (a) an aliphatic hydrocarbyl group of from 1 to about 22 carbon atoms or (b) hydrogen or an aromatic, alkoxy, polyoxyalkylene, alkylamido, hydroxyalkyl, aryl or alkylaryl group having up to about 22 carbon atoms; and X is a salt-forming anion such as those selected from halide (e.g., chloride, bromide), acetate, citrate, lactate, glycolate, phosphate, nitrate, sulfate, and alkylsulfate radicals, wherein at least one of R¹, R², R³, and R⁴ is an aliphatic hydrocarbyl group having from about 8 to about 22 carbon atoms, preferably alkyl. The aliphatic hydocarbyl groups can contain, in addition to carbon and hydrogen atoms, ether linkages, and other groups such as amino groups. The longer chain aliphatic hydrocarbyl groups, e.g., those of about 12 carbons, or higher, can be saturated or unsaturated. Salt-forming anions of particular interest are halides, preferably chloride. The cationic conditioning surfactants may be used singly or in admixture.

Included among the suitable cationic conditioning surfactants are mono- and dialkyl quats. Monoalkyl quats suitable for use herein include Formula (III) conditioning surfactants wherein R¹, R², and R³, which may be the same or different, are C₁-C₄ alkyl groups (preferably ethyl or methyl) and R⁴ is a C₈ or greater aliphatic hydrocarbyl group (preferably C₁₄ to C₂₂ alkyl). Other monoalkyl quats suitable for use herein are Formula (III) conditioning surfactants wherein R¹ and R², which may be the same or different, are C₁-C₄ alkyl groups (preferably ethyl or methyl), R³ is an aryl group, preferably benzyl, and R⁴ is a C₈ or greater aliphatic hydrocarbyl group (preferably C₁₄ to C₂₂ alkyl). The monoalkyl quats may, but need not, be in the form of mixtures. Non-limiting examples of monoalkyl quats are:

cetyltrimethylammonium chloride (C16);

stearyltrimethylammonium chloride (C18);

behenyltrimethylammonium chloride (C22);

cetyltrimethylammonium bromide (C16);

tallowtrimonium chloride (C16/C18);

behenyltrimethylammonium methosulfate (C22);

palmityltrimethylammonium chloride (C16);

hydrogenated tallowtrimethylammonium chloride (C16/C18);

hydrogenated tallowtrimethylammonium bromide (C16/C18);

hydrogenated tallowtrimethylammonium methosulfate (C16/C18);

cetrimonium tosylate (C16): and

eicosyltrimethylammonium chloride (C20).

Also included among the Formula (III) conditioning surfactants are dialkyl quats in which R¹ and R², which may be the same or different, are C1-C4 alkyl groups and R³ and R⁴, which may be the same or different, are C8 or greater aliphatic hydrocarbyl groups (preferably C14 to C22 alkyl). If desired, the dialkyl quats may be in the form of mixtures. Non-limiting examples of dialkyl quats are:

dimethyldicetylammonium chloride (C16);

dimethyldistearylammonium chloride (C18);

dimethyldipalmitylammonium chloride (C16);

dimethyl(dihydrogenatedtallow)ammonium chloride (C16/C18);

dimethyl(ditallow)ammonium chloride (C16/C18)

dimethyl(dihydrogenatedtallow)ammonium bromide (C16/C18)

dimethyl(dihydrogenatedtallow)ammonium methosulfate (C16/C18)

In at least one embodiment of this invention, dialkyl quats of particular interest include mixtures of C16 and C18 dialkyl quats.

Desirably, the cationic conditioning surfactant is present in the preshampoo conditioner in amounts of from about 0.1 to about 5 percent by weight, based on the total weight thereof. In many applications, pre-shampoo conditioners wherein the cationic conditioning surfactant is present in an amount of from 0.2 to 3 percent, more particularly, from 0.5 to 2 percent by weight, based on the total weight of the pre-shampoo conditioner, are of particular interest. In the practice of this invention, cationic conditioning surfactants comprising mixtures of monoalkyl quats and dialkyl quats wherein the ratio of the monoalkyl quat to dialkyl quat is from 15:1 to 1:0.5, more particularly from 0:1 to 1:1 are of particular interest. In one preferred embodiment, the Formula (III) cationic conditioning surfactant is a mixture of monoalkyl quats and dialkyl quats wherein the ratio of monalkylquat to dialkylquat ranges from 5:1 to 1:1. Of particular interest are mixtures of C16 to C22 mono- and dialkyl quats, for example, cetyltrimethylammonium chloride in combination with dimethyl(dihydrogenated tallow)ammonium chloride.

As used herein the term “fatty alcohol material” means a fatty alcohol, an alkoxylated fatty alcohol, or a mixture thereof. The fatty alcohol component of the pre-shampoo conditioner comprises from about 8 to about 22 carbon atoms, more preferably about 12 to about 20 carbon atoms. Examples of preferred fatty alcohols are cetyl alcohol, stearyl alcohol and mixtures thereof. Alkoxylated, (e.g., ethoxylated or propoxylated) fatty alcohols can be used in place of or in addition to, the fatty alcohols themselves. Additional examples of suitable fatty alcohol materials include ethylene glycol ethyl ether, polyoxyethylene (2) stearyl ether, polyoxyethylene cetyl ether, and mixtures thereof.

Without wishing to be bound to theory it is believed that fatty alcohol material can form a lamellar phase in which the cationic components are dispersed, enabling uniform distribution thereof. The level of fatty alcohol material is suitably 2 to 15 percent by weight, more particularly from 2 to 10 percent by weight, based on the total weight of the pre-shampoo conditioner.

One or more additional ingredients as are conventionally incorporated into hair conditioning compositions may be included in the pre-shampoo conditioners of the invention. Such additional ingredients include, for example, fragrances, perfumes, dyes, coloring agents, buffering or pH adjusting agents, chelating agents, thickening agents, suspending agents, viscosity modifiers, opacifiers, pearlizers, preservatives, antibacterial agents, proteins, amino acids, moisturizing agents, herb or other plant extracts and other natural ingredients.

The shampoo component of subject system comprises an anionic surfactant, at least one cationic conditioning agent, and water. Suitable anionic surfactants include, for example, alkyl sulfates, alkyl ether sulfates, alkaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di-, and triethanolamine salts, and mixtures thereof. The alkyl and acyl groups generally contain from 8 to 18 carbon atoms and may be unsaturated. The alkyl ether sulfates, alkyl ether phosphates and alkyl ether carboxylates may contain from 1 to 10 ethylene oxide (EO) or propylene oxide units per molecule, and preferably contain 2 to 3 ethylene oxide units per molecule. Of particular interest in the practice of this invention are anionic surfactants selected from the group consisting of sodium lauryl sulfate, triethanolamine lauryl sulfate, triethanolamine monolauryl phosphate, sodium lauryl ether sulfate 1 EO, 2EO and 3 EO, ammonium lauryl sulfate and ammonium lauryl ether sulfate 1 EO, 2EO and 3 EO, and mixtures thereof. In an embodiment of particular interest, the preferred anionic surfactant is sodium lauryl ether sulfate. Desirably, the anionic surfactant is present in the shampoo component of this invention in an amount of from 5 to 20 percent by weight, based on the total weight of the shampoo composition, with shampoo compositions wherein the anionic surfactant is present in an amount of from 8 to 18 percent by weight, more preferably from 10 to 16 percent by weight based on the total weight of the shampoo composition, being of particular interest.

Optionally, the shampoo may comprise one or more additional surfactants. These additional surfactants include nonionic, amphoteric and/or zwitterionic surfactants. Suitable nonionic surfactants include condensation products of aliphatic (8 to 20 carbons) primary, secondary linear or branched chain alcohols or phenols with alkylene oxides, usually ethylene oxide and generally having from 6 to 30 ethylene oxide groups. Other suitable nonionics include alkanolamides such as, for example, coco mono-diethanolamide, coco mono-isopropanolamide, and mixtures thereof. Amphoteric and zwitterionic surfactants suitable for use in compositions of the invention include, for example, alkyl amine oxides, alkyl betaines, alkyl amidopropyl betaines, alkylsulfobetaines, alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 20 carbon atoms. Examples include lauryl amine oxide, cocodimethyl sulphopropyl betaine, lauryl betaine, cocamidopropyl betaine, sodium cocamphopropionate, and mixtures thereof. The additional surfactants are present in an amount up to 20% by weight, preferably from 0.5 to 20% by weight, more preferably from 0.5 to 10% based on the total weight of the shampoo.

Exemplary of the cationic conditioning agents suitable for use in the shampoo component of the subject invention are quaternary silicone polymers, quaternary polysaccharide polymers, quaternary cellulose derivatives, quaternary starch derivatives and quaternary guar gum derivatives. Exemplary of the quaternary silicone polymer are materials of the general formula (IV):
R¹R²R³N⁺-A-SiR⁷R⁸—(O—SiR⁹R¹⁰)ₙ—O—SiR¹¹R¹²-A-N⁺R⁴R⁵R⁶2X⁻ (IV)
wherein R¹ to R⁶ are independently an unsubstituted C1 to C22 alkyl group or a substituted C1 to C22 alkyl group with at least one hydroxyl group; R⁷ to R¹² are independently a C1 to C10 alkyl group or a phenyl group; A is a divalent organic compound group, n is a number from 0 to 200, preferably from 10 to 120, and, in at least one embodiment of this invention, from 10 to 40, and X⁻ is an anion. The divalent compound group A is preferably a C1 to C12-alkylene or alkoxyalkylene group, which can optionally be substituted with one or more hydroxyl groups. Formula (IV) polymers wherein A is —(CH₂)₃—O—CH₂—CH(OH)—CH₂— are especially preferred. The anion X⁻ can be a halogen ion, an acetate, an organic carboxylate or a compound of the general formula RSO₃⁻, wherein R is a C1-alkyl to C4-alkyl group. In at least one embodiment of the invention, preferred quaternary silicone polymer compounds are materials of the general formula (V):
R—N⁺Me₂-A-(SiMe₂O)ₙ^{—}SiMe₂-A-N⁺Me₂R₂CH₃COO⁻ (V)
wherein A is —(CH₂)₃—O—CH₂—CH(OH)—CH₂—, R is an alkyl group with at least eight carbon atoms and n is a number from 10 to 120.

Suitable silicone polymer compounds with two terminal quaternary ammonium groups are known under the INCI name, Quaternium-80. They are dimethylsiloxanes with two terminal trialkylammonium groups. These diquaternary polydimethylsiloxanes are marketed by Goldschmidt under the trademark Abil Quat 3270, Abil Quat 3272, and Abil Quat 3474.

Quaternary polysaccharide polymers suitable for use in the compositions of the invention include those of the formula: 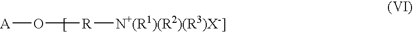
wherein A is an anhydroglucose residual group such as a starch or cellulose anhydroglucose residue, R is an alkylene, oxyalkylene, polyoxyalkylene, or hydroxyalkylene group, or combinations thereof. R¹, R², and R³ independently represent alkyl, aryl, alkaryl, aryalkyl, alkoxyalkyl or alkoxyaryl groups, each group containing up to about 18 carbon atoms. The total number of carbon atoms for each quaternary moiety (i.e., the sum of carbon atoms in R¹, R², and R³) is preferably about 20 or less, and X is an anionic counterion.

Quaternary cellulose derivatives are available from Amerchol Corp. in their Polymer JR and LR series of polymers, as salts of hydroxyethyl cellulose reacted with trimethyl ammonium substituted epoxide, referred to in the industry (CTFA) as Polyquaternium 10. Another type of quaternary cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp (Edison, N.J., USA) under the tradename Polymer LM-200.

Quaternary cellulose ethers (e.g., as described in U.S. Pat. No. 3,962,418), and copolymers of etherified cellulose and starch (e.g., as described in U.S. Pat. No. 3,958,581) are also suitable for use herein. Suitable quaternary guar gum derivatives include, for example guar hydroxypropyltrimonium chloride (commercially available from Rhone-Poulenc in their Jaguar series of products).

In at least one embodiment of interest the quaternary conditioning agent comprises a quaternary silicone polymer. In another embodiment of interest the quaternary conditioning agent comprises a quaternary silicone polymer and at least one additional quaternary silicone agent selected from quaternary polysaccharide polymers, quaternary cellulose derivatives, quaternary starch derivatives and quaternary guar gum derivatives.

The cationic conditioning agent may be incorporated into the subject shampoos in an amount of from 0.05 to 2 percent by weight, preferably from 0.1 to 2 percent by weight, and, more preferably, from 0.1 to 1.5 percent by weight, based on the total weight of the shampoo composition. In one embodiment of particular interest, the quaternary conditioning agent is present in an amount of from 0.1 to 1 percent by weight, based on the total weight of the shampoo composition; in another embodiment of particular interest the quaternary conditioning agent is present in an amount of from 0.2 to 0.8 percent by weight, based on the total weight of the shampoo composition. The amount of cationic conditioning agent is exclusive of any suspending agents, solvents, or carriers, present in such component, i.e., the amount of cationic conditioning agent is reported on an active basis.

While quaternary silicone polymers may be present in shampoo compositions used in the practice of this invention, in one preferred embodiment, the shampoo compositions are “substantially free” of substantive conditioner that with repeated shampoo use can lead to a “weight down” or greasy appearance in fine hair. Additionally, in at least one embodiment of this invention, the shampoo is substantially free of organopolysiloxanes such as dimethicone, amodimethicone, dimethiconal, and the like (referred to as “organopolysiloxane(s)”). As used herein “substantially free” means an amount not to exceed 1 percent by weight, based on the total weight of the shampoo composition. In one embodiment of interest, the amount of organopolysiloxane present in the shampoo composition does not exceed 0.7 percent by weight.

One or more additional ingredients as are conventionally incorporated into shampoos may be included in compositions of the invention. Such additional ingredients include, for example, fragrances, perfumes, dyes, coloring agents, buffering or pH adjusting agents, deposition aids, chelating agents, viscosity modifiers, thickening agents, suspending agents, opacifiers, pearlizers, antidandruff agents, sunscreens, antioxidants, preservatives, antibacterial agents, proteins, amino acids, moisturizing agents, herb or other plant extracts and other natural ingredients.

The preshampoo conditioners and shampoos of this invention may be prepared by conventional techniques known to those skilled in the art, for example, emulsification under conditions of high shear at elevated temperatures.

In use, the pre-shampoo conditioner is applied and massaged into hair that to promote ease of spreading, has desirably been moistened prior to the conditioner's application. The pre-shampoo conditioner may, however also be applied while the hair is dry. Treatment with the pre-shampoo conditioner, which is preferably worked into the hair by gentle massaging, will typically be completed within 5 minutes or less, typically 1-2 minutes. Thereafter, the hair is cleaned with the shampoo, rinsed with water and dried. Preferably, the pre-shampoo conditioner is rinsed from the hair prior to application of the shampoo to remove excess conditioner and promote better cleaning of the hair, however, the shampoo may also be applied while the pre-shampoo conditioner is still on the hair.

The pre-shampoo conditioner and shampoo components of the subject systems are packaged as separate components of a single unit or kit with instructions regarding their use. The term “individually packaged” means that the contents of the pre-shampoo conditioner package are maintained separate and distinct from that of the pre-shampoo condition. While kits comprising separate, stand-alone, containers are within the scope of the subject invention, the use of such stand-alone containers is not required. For example, the pre-shampoo conditioner and shampoo may be contained in separate chambers of a single multi-chamber container. Alternatively, the packages of shampoo and conditioner may be separate containers that are joined together, such as for, example, in interlocking fashion. The use of kits wherein the packaging for the pre-conditioner and shampoo components takes the form of a single integrated container serve as a reminder for joint usage and also educate the consumer in the proper application thereof. The subject kits may be formulated for single or repeated use.

Desirably, the kit should contain pre-shampoo conditioner and shampoo in relative amounts such that consumers do not run out of one component long before the other. Hair condition, shampooing habits and hair length are factors that affect the relative amounts of pre-shampoo conditioner and shampoo that consumers use. For many consumers, kits wherein the ratio, by volume, of shampoo to pre-shampoo conditioner is in a range from 1:1 to 2:1: will provide amounts of these components that approximate their relative rate of use.

The invention is further illustrated by way of the following non-limiting examples. Unless otherwise indicated, all parts and percentages are by weight.

### EXAMPLES

### Example 1

Pre-shampoo conditioners were formulated by preparing emulsions having the formulations described in Table 1.

Charge Density

The charge densities of the polyquaternium components used in formulating the conditioners were as follows:

Polyquaternium 6—2.22 meq/g

Polyquaternium 7—0.64 meq/g

Polyquternium 32—1.87 meq/g

Polyquaternium 37—2.27 meq/g

Charge densities were measured by means of the following procedure, referred to here and elsewhere in this document as “anionic potentiometric titration testing.”

In this procedure an approximately 0.1 g sample of the cationic material, the charge density of which is to be measured, is accurately weighed into a 250 mL titration beaker and the weight of the sample is recorded. 100 mL of deionized water, followed by 2 ml of a 1% aqueous solution of Titron X-100 electrode cleaning solution are added to the beaker. A surfactant sensitive electrode, Orion Model 93-42 or equivalent, is preconditioned in a 0.0001 N aqueous solution of sodium lauryl sulfate (SLS) and rinsed clean with deionized water, immersed in the titration beaker and allowed to stabilize before performing the titration. The sample is then titrated with a standardized 0.02N SLS solution and the volume of titrant added to reach the potentiometric endpoint is recorded.

Charge density is calculated by means of the following formula:
Charge density (meq/g)=(V)(N_{SLS})(1000)/W

N_{SLS}=the standardized concentration of the SLS solution used in the titration (eq/L)

W=the sample weight (mg)

V=the volume of the SLS titrant (mL)

The SLS solution is standardized by using it to titrate a solution of 0.075±0.005 g of benethionium chloride in 75 ml of deionized water, using a surfactant sensitive electrode as described above. The normality of the SLS solution (eq/L) is determined by means of the following equation:
N=(W_{BC})(Purity_{BC})/(V_{SLS})(448.1 g/eq)
where:

W_{BC}=the weight of the benethionium chloride sample (g)

Purity_{BC}=the percent purity of the benethionium chloride sample/100

V_{SLS}=the volume of the SLS titrant (L)

448.1 g/eq=the equivalent weight of the benethionium chloride

N_{SLS} is the average normality of the SLS solution determined on triplicate analyses, where an accuracy of ±0.0005N is obtained between replicates.

In measurements used to obtain the charge densities reported above, the benethionium chloride used in the standardization of the SLS solution was Hyamine 1622 (99% purity).

TABLE 1
Pre-shampoo Conditioner Formulations

| | FORMULATION | | | | | | | | |
|---|---|---|---|---|---|---|---|---|---|
| | A1 | A2 | A3 | A4 | A5 | A6 | A7 | A8 | A9 |
| Component | Weight % | | | | | | | | |
| Cetrimonium chloride | 2.50 | 2.50 | 2.50 | 2.50 | 2.50 | 2.50 | 2.50 | 2.50 | 2.50 |
| (30% active in water) | | | | | | | | | |
| Quaternium 18 (75% active | 0.40 | 0.40 | 0.40 | 0.40 | 0.40 | 0.40 | 0.40 | 0.40 | 0.40 |
| in propylene glycol) | | | | | | | | | |
| Cetearyl alcohol | 3.00 | 3.00 | 3.00 | 3.00 | 3.00 | 3.00 | 3.00 | 3.00 | 3.00 |
| Potassium chloride | 0.25 | 0.25 | 0.25 | 0.25 | 0.25 | 0.25 | 0.25 | 0.25 | 0.25 |
| Disodium EDTA | 0.10 | 0.10 | 0.10 | 0.10 | 0.10 | 0.10 | 0.10 | 0.10 | 0.10 |
| Preservative | 0.14 | 0.14 | 0.14 | 0.14 | 0.14 | 0.14 | 0.14 | 0.14 | 0.14 |
| Fragrance | 0.60 | 0.60 | 0.60 | 0.60 | 0.60 | 0.60 | 0.60 | 0.60 | 0.60 |
| Polyquaternium-6 (40% active) | 4.00 | 3.00 | 1.50 | — | — | — | — | — | — |
| Polyquaternium-7 (40% active) | — | — | — | — | — | — | — | — | 3.00 |
| Polyquaternium-32 (50% active) | — | — | — | — | — | — | 2.40 | 1.20 | — |
| Polyquaternium-37 (50% active) | — | — | — | 3.20 | 2.40 | 1.20 | — | — | — |
| Deionized water | balance | balance | balance | balance | Balance | balance | balance | Balance | balance |
| | to 100 | to 100 | to 100 | to 100 | to 100 | to 100 | to 100 | to 100 | to 100 |

Wet Combing Performance

In this procedure, test specimens were 2 g tresses of bleached and waved hair. 0.3 ml of the pre-shampoo conditioner being evaluated was massaged into the tresses for 1 minute; the tresses were then rinsed with soft water for 30 seconds. Following rinsing, each tress was shampooed with 0.2 ml of Suave® Aloe Vera Shampoo for 1 minute; the shampooed tresses were then rinsed with soft water for 30 seconds. As a first control, tresses were treated with 0.2 ml Suave Aloe Vera Shampoo (1 minute) followed by rinsing with soft water for 30 seconds (no conditioner treatment); as a second control, tresses were first treated with 0.2 ml of Suave Aloe Vera Shampoo (1 minute) followed by rinsing with soft water for 30 seconds, the rinsed, shampooed tresses were then treated with 0.3 ml of Finess® Extra Moisturizing Conditioner (1 minute) followed by rinsing with soft water (30 seconds).

Prior to wet combing, excess moisture was removed by blotting the rinsed tresses with paper towels. The tresses were then subjected to wet combing using an Instron 5500 series tensile tester. Three measurements were obtained from each tress, and the procedure was repeated such that three tresses were tested for each system evaluated. The maximum load (highest force encountered during combing) is reported in Table 2 as an average of nine measurements (i.e, 3 measurements from each of 3 tresses). As demonstrated by the Table 2 data, hair treated with pre-shampoo conditioners A1 to A9 all had improved conditioning performance compared to Control 1 (hair that was shampooed but had no conditioning treatment. Hair treated with the preshampoo conditioners containing the higher charge density polyquaterniums, i.e., Polyquaternium 6, Polyquaternium 32 or Polyquaternium 37, had better conditioning performance than hair treated with the conditioner containing the lower charge density Polyquaternium 7. Hair treated with pre-shampoo conditioners A1 and A2, both of which contained Polyquaternium 6, exhibited a mean load of 14.5 gm force and 15.0 gm force, respectively and came closest to the wet combing performance of the hair treated with after-shampoo conditioner (Control 2).

TABLE 2
Wet Combing Data

| | Max Load |
|---|---|
| FORMULATION | (gm Force) |
| A1 | 14.5 |
| A2 | 15.0 |
| A3 | 21.6 |
| A4 | 17.9 |
| A5 | 17.8 |
| A6 | 20.4 |
| A7 | 21.5 |
| A8 | 25.7 |
| A9 | 37.3 |
| Control 1- Shampoo only - no conditioner treatment. | 45.3 |
| Control 2 -Shampoo, followed by treatment with | 8.3 |
| Finesse ® Extra Moisturizing Conditioner | |

### Example 2

Sample Preparation

A pre-shampoo conditioner containing Polyquaternium-6 was prepared according to the formulation described in Table 3 (pre-shampoo conditioner formulation A10).

Shampoo formulations were prepared according to the formulations described in Table 4.

B6 was a commercially available moisturizing shampoo comprising anionic surfactant, quaternary conditioning agent and approximately 1.5% of a combination of dimethiconol and amodimethicone.

TABLE 3
Pre-Shampoo Conditioner Formulation (A10)

| Component | Weight Percent |
|---|---|
| Cetrimonium chloride (30% active) | 2.50 |
| Quaternium 18 in propylene glycol (75% active) | 0.40 |
| Cetearyl/alcohol | 3.00 |
| Potassium chloride | 0.25 |
| Disodium EDTA | 0.10 |
| Preservative | 0.14 |
| Fragrance | 0.60 |
| Polyquaternium-6 | 2.5 |
| Water | balance to 100 |

TABLE 4
Shampoo Formulations

| | FORMULATION | | | | |
|---|---|---|---|---|---|
| Component (wt. %) | B1 | B2 | B3 | B4 | B5 |
| Deionized water | to 100 | to 100 | to 100 | to 100 | to 100 |
| Abil 3474 | — | 0.50 | — | — | — |
| (50% active) | | | | | |
| Crodacel QM | 2.00 | — | — | — | — |
| (20% active) | | | | | |
| Polymer JR | — | — | — | 0.10 | 0.20 |
| Cocamidopropyl | 5.71 | 5.71 | 5.71 | 5.71 | 5.71 |
| betaine | | | | | |
| (28% active) | | | | | |
| SLES-1 | 56.00 | 56.00 | 48.00 | 48.00 | 48.00 |
| (25% active) | | | | | |
| Fragrance | 0.8 | 0.80 | 0.80 | 0.80 | 0.80 |
| Jaguar Excel | 0.15 | 0.15 | 0.15 | — | — |
| (98% active) | | | | | |
| Preservative | 0.14 | 0.14 | 0.14 | 0.14 | 0.14 |
| Tetrasodium EDTA | 0.20 | 0.20 | 0.20 | 0.20 | 0.20 |
| (39% active) | | | | | |
| Dimethiconol | — | — | 2.00 | — | 2.00 |
| microemulsion | | | | | |
| (25% active) | | | | | |
| Amodimethicone | — | — | 1.00 | — | — |
| microemulsion | | | | | |
| (20% active) | | | | | |
| Citric acid | 0.06 | 0.06 | — | 0.13 | 0.12 |
| (50% active) | | | | | |
| Sodium hydroxide | — | — | 0.15 | — | — |
| (50% active) | | | | | |
| Ammonium chloride | 0.10 | 1.30 | 1.55 | 1.10 | — |

Salon Testing

The performance of pre-shampoo conditioner A10 in conjunction with a shampoo selected from formulations B1 to B6 was evaluated in a series of salon tests. The tests were conducted by stylists who applied the pre-shampoo conditioner (PSC) and shampoo following the protocol set forth below. Salon testing results are reported in Table 5.

Protocol

The test subjects were females who, through screening, were identified as body/volume seekers.

The protocol was as follows:
1) both sides of head were wet;
2) the PSC was applied to both sides of the head and massaged into the hair for 60 seconds;
3) the hair was rinsed thoroughly;
4) a first shampoo was applied to one side of the head and massaged into the hair on that side for about ½ to 1 minute; a second shampoo was applied to the other side of the head and massaged into the hair on that side for about ½ to 1 minute;
5) the shampooed hair on each side of the head was rinsed. After all products were rinsed from the hair, the test subjects evaluated wet and dry attributes for each side of their head.

Product quantities used for shampoo and PSC: ˜3 ml for hair chin length or shorter, ˜5 ml for chin to shoulder length hair, ˜2 ml for hair longer than shoulder length.

Test Design

In the panel where two different shampoos were evaluated, a Balanced Complete Block (BCB) design was used. In the panels where three or four shampoos were evaluated, a Balanced Incomplete Block (BIB) design was used (e.g. 2 out of 4 BIB design for 4 products). Samples were randomized so as to promote balance with respect to shampoo samples being seen equally often and on the left and right sides of the head equally often.

TABLE 5
Salon Test Results

| | Shampoo Formulation | | | | | | | | |
|---|---|---|---|---|---|---|---|---|---|
| | B4 | B5 | B3 | B6 | B4 | B1 | B6 | B4 | B2 |
| Panel/No. of Participants | 1/40 | 1/40 | 1/40 | 1/40 | 2/40 | 2/40 | 2/40 | 3/44 | 3/44 |
| Wet Stage | | | | | | | | | |
| Ease of detangling | 7.8 bB | 8.3 aA | 8.3 aA | 8.2 aAB | 7.1 C | 7.7 B | 8.2 A | 7.7b | 8.1 a |
| Coated feel | 5.0 B | 4.6 B | 4.5 B | 5.8 A | 4.3 | 4.9 | 4.5 | 5.1 | 4.8 |
| Clean feel | 7.4 bAB | 7.3 bB | 7.9 aA | 7.6 abAB | 7.2 | 7.3 | 7.5 | 7.2 | 7.3 |
| Amount of conditioning | 6.2 B | 6.2 B | 6.3 B | 7.1 A | 6.1 B | 6.6 A | 6.9 A | 6.6 | 6.7 |
| Dry Stage | | | | | | | | | |
| Ease of combing/brushing | 7.7 cB | 8.0 bcAB | 8.3 aA | 8.3 abA | 6.9 B | 7.7 A | 8.0 A | 7.5 | 7.8 |
| Clean feel | 7.4 AB | 7.0 B | 7.8 A | 7.8 A | 7.3 | 7.5 | 7.6 | 7.2B | 7.6 A |
| Amount of conditioning | 6.4 ab | 6.1 b | 6.6 ab | 6.8 a | 6.4 | 6.3 | 6.8 | 6.4 | 6.8 |
| Ease of Styling | 6.4 b | 6.8 ab | 7.0 a | 6.7 ab | 7.1 | 6.9 | 7.1 | 6.9b | 7.3 a |
| Amount of manageability | 6.0 b | 6.4 ab | 6.5 a | 6.3 ab | 6.8 | 6.4 | 7.0 | 6.7 | 6.8 |
| Amount of volume fullness | 5.9 bB | 6.1 bAB | 6.7 aA | 6.2 abAB | 6.8 | 7.1 | 6.7 | 6.2B | 6.8 A |
| Amount of bounce | 6.2 AB | 5.9 B | 6.6 A | 6.3 AB | 6.6 | 6.8 | 6.3 | 6.3B | 6.9 A |
| Amount of body | 6.0 B | 5.9 B | 6.9 A | 6.3 AB | 6.6 b | 7.1a | 6.6 b | 6.2B | 7.0 A |
| Amount of static | 3.8 | 3.3 | 3.7 | 3.9 | 4.3 | 4.9 | 4.4 | 4.3 | 4.5 |

Shared letters for a given test result means that the result is not statistically different at the 90% (small letter) or 95% (capital letter) confidence level. A lack of statistical difference is also indicated by results that do not report any letters.

The salon data was based on one-time usage. To evaluate performance over time, microscopy testing was employed using hair tress samples subjected to repeated pre-shampoo conditioner/shampoo treatments.

Microscopy Testing

Following the preshampoo conditioner/shampoo treatment procedure described in Example 1, 2 g hair tresses (two tresses per system) were treated with pre-shampoo conditioner A10 followed by one of four different shampoo compositions (B1, B2, B3 or B6). The procedure was repeated to provide one tress per system with five consecutive preshampoo conditioner/shampoo treatments. Hair treated with only a non-conditioning stripping shampoo (no pre-shampoo conditioner), was employed as a control. A scanning electron microscope was used to evaluate buildup of conditioner on the hair fibers. Microscopy testing was conducted as follows:

Fibers from each tress were mounted onto aluminum stubs using double-sided carbon adhesive tape. A thin layer of platinum was then deposited over each sample using an Emitech K-575 Turbo Sputter Coater to minimize charging of the fibers while under the electron beam of the scanning electron microscope (SEM). Tresses were then analyzed using a Hitachi s-4500 Field Emission SEM, operated at an accelerating voltage of 5 kV. Images were taken at magnification between 1300× to 6000×. The images were examined and rated for surface deposition, using a scale of 0 to 5. Microscopy results are reported in Table 6.

TABLE 6
Microscopy Results

| Preshampoo | Relative | |
|---|---|---|
| Conditioner/Shampoo | Rating | Surface Deposit Description |
| Control | 0 | No deposits |
| A10/B2 - 1 treatment | 0 | Close to untreated |
| A10B2 - 5 treatments | 2 | Light deposits |
| A10/B1 - 1 treatment | 0 | Close to untreated |
| A10/B1 - 5 treatments | 2 | Light deposits |
| A10/B3 - 1 treatment | 1 | Very light deposits |
| A10/B3 - 5 treatments | 3 | Moderate deposits |
| A10/B6 - 1 treatment | 2 | Light deposits |
| A10/B6 - 5 treatment | 5 | Highest build-up of deposits of |
| | | compositions evaluated |

What is claimed is:

1. A system for treating and conditioning hair which system comprises, as individually packaged compositions:
(A) an aqueous pre-shampoo conditioner comprising:
i) a cationic conditioning polymer having a charge density of at least 1.8 meq/g,
ii) a cationic conditioning surfactant,
iii) a fatty alcohol material; and
(B) an aqueous shampoo comprising:
i) an anionic surfactant, and
ii) a cationic conditioning agent selected from the group consisting of quaternary silicone polymers, quaternary polysaccharide polymers, quaternary cellulose derivatives, quaternary starch derivatives, quaternary guar gum derivatives, and mixtures thereof.

2. A system as described in claim 1 which includes instructions to apply the shampoo to the hair after rinsing the pre-shampoo conditioner from the hair and while the hair is at least damp.

3. A system as described in claim 1 wherein the cationic conditioning polymer comprises a polymer having units of the formula: 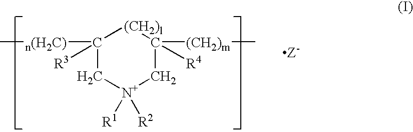
wherein R¹ and R², which may be the same or different, represent hydrogen or an alkyl group having 1-18 carbon atoms, R³ and R⁴, which may be the same or different, represent hydrogen, an alkyl group having 1-3 carbon atoms or a phenyl group, and l and m and n are integers having a value of 0 or 1, with the proviso that at least one of m or n is 1; and Z is a salt-forming anion.

4. A system as described in claim 1 wherein the cationic conditioning polymer comprises a polymer having units of the formula: 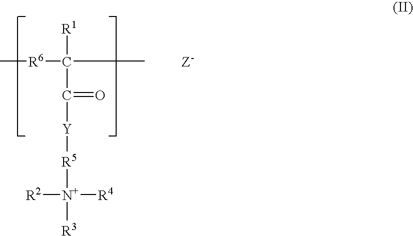
wherein R¹, stands for a hydrogen atom or an alkyl group having 1 to 3 carbon atoms, R², R³ and R⁴, which may be the same or different, stand for hydrogen, an alkyl group having 1 to 3 carbon atoms or a phenyl group, R⁵ is a divalent aliphatic hydrocarbyl group having from about 2 to about 6 carbon atoms, and R⁶ is a divalent aliphatic hydrocarbyl group having from about 1 to 6 carbon atoms which is optionally substituted with ester and/or ester amide linkages, Y is O or N(R⁷) where R⁷ is a hydrogen atom or an alkyl group having 1 to 3 carbon atoms; and Z is a salt-forming anion.

5. A system as described in claim 1 wherein the cationic conditioning polymer comprises Polyquaternium-6.

6. A system as described in claim 1 wherein the cationic conditioning polymer comprises Polyquaternium-37.

7. A system as described in claim 1 wherein the cationic conditioning polymer has a charge density of from 2 meq/g to 3 meq/g.

8. A system as described in claim 1 wherein the cationic conditioning surfactant comprises at least one compound of the formula:
[N(R¹)(R²)(R³)(R⁴)]⁺[X]⁻ (III)
wherein R¹, R², R³, and R⁴ are independently selected from (a) an aliphatic hydrocarbyl group of from 1 to about 22 carbon atoms or (b) hydrogen or an aromatic, alkoxy, polyoxyalkylene, alkylamido, hydroxyalkyl, aryl or alkylaryl group having up to about 22 carbon atoms; and X is a salt-forming anion, and wherein at least one of R¹, R², R³, and R⁴ is an aliphatic hydrocarbyl group having from about 8 to about 22 carbon atoms

9. A system as described in claim 8 wherein the cationic conditioning surfactant comprises at least one mono-alkyl quat.

10. A system as described in claim 1 wherein the cationic conditioning agent is present in the shampoo in an amount of from 0.1 to 2 percent by weight.

11. A system as described in claim 1 wherein the cationic conditioning agent is present in the shampoo in an amount of from 0.2 to 0.8 percent by weight.

12. A system as described in claim 1, wherein the system further comprises packaging that identifies the system as appropriate for users seeking hair with body or volume.

13. A system as described in claim 1 wherein the shampoo is substantially free of conditioning agents that weigh down the hair.

14. A system as described in claim 1 wherein the shampoo further comprises one or more organopolysiloxanes in an amount that does not exceed 0.7 percent by weight.

15. A system for treating and conditioning hair which system comprises, as individually packaged compositions:
(A) an aqueous pre-shampoo conditioner comprising:
i) a cationic conditioning polymer having a charge density of at least 1.8 meq/g,
ii) a cationic conditioning surfactant, and
iii) a fatty alcohol material; and
(B) an aqueous shampoo comprising:
i) an anionic surfactant, and
ii) a cationic conditioning agent selected from the group consisting of quaternary silicone polymers, quaternary polysaccharide polymers, quaternary cellulose derivatives, quaternary starch derivatives, quaternary guar gum derivatives and mixtures thereof.

16. A method for cleaning and conditioning hair that comprises the sequential steps of:
(A) treating the hair with an aqueous pre-shampoo conditioner comprising:
i) a cationic conditioning polymer having a charge density of at least 1.8 meq/g,
ii) a cationic conditioning surfactant,
iii) a fatty alcohol material; and
(B) treating the hair with an aqueous shampoo comprising:
i) an anionic surfactant, and
ii) a cationic conditioning agent selected from the group consisting of quaternary silicone polymers, quaternary polysaccharide polymers, quaternary cellulose derivatives, quaternary starch derivatives, quaternary guar gum derivatives and mixtures thereof.

17. A method for cleaning and conditioning hair as described in claim 16 wherein the cationic conditioning polymer comprises Polyquaternium-6.

18. A method for cleaning and conditioning hair as described in claim 16 wherein the cationic conditioning polymer comprises Polyquaternium-37.

19. A system for treating and conditioning hair as described in claim 1 wherein the pre-shampoo conditioner consists essentially of said cationic conditioning polymer, said cationic conditioning surfactant, and said fatty alcohol material.
